(19) United States
(12) Patent Application Publication
(10) Pub. No.: US 2021/0307728 A1
(43) Pub. Date: Oct. 7, 2021

(54) ULTRASOUND IMAGING DEVICE, ULTRASOUND IMAGING SYSTEM, OPERATION METHOD FOR ULTRASOUND IMAGING DEVICE, AND COMPUTER READABLE RECORDING MEDIUM

(71) Applicant: OLYMPUS CORPORATION, Tokyo (JP)

(72) Inventor: Kei TAGUCHI, Tokyo (JP)

(73) Assignee: OLYMPUS CORPORATION, Tokyo (JP)

(21) Appl. No.: 17/351,585

(22) Filed: Jun. 18, 2021

Related U.S. Application Data
(63) Continuation of application No. PCT/JP2019/000246, filed on Jan. 8, 2019.

(51) Int. Cl. A61B 8/00; A61B 8/12

(52) U.S. Cl. CPC A61B 8/54; A61B 8/12; A61B 8/4245

(57) ABSTRACT

An ultrasound imaging device includes: a processor including hardware, the processor being configured to control ah ultrasound transducer to transmit a push pulse in response to input of an operation; set, based on an amount of movement of the ultrasound transducer in a predetermined time period according to the input of the operation, transmission parameter for transmitting a track pulse; and control the ultrasound transducer to transmit the track pulse toward a shear wave detection position set in an ultrasound image.

This application is a continuation of International Application No. PCT/JP2019/000246, filed on Jan. 8, 2019, the entire contents of which are incorporated herein by reference.

### BACKGROUND

The present disclosure relates to an ultrasound imaging device, an ultrasound imaging system, an operation method for the ultrasound imaging device, and a computer readable recording medium.

Ultrasound imaging devices for generating ultrasound images based on ultrasound signals acquired through transmission and reception of ultrasound by ultrasound transducers, to and from subjects, which are observation targets, have been used in the medical field.

These ultrasound imaging devices include ultrasound imaging devices that set a region of interest (ROI) in an ultrasound image, generate shear waves by transmission of push pulses to the region of interest, transmit and receive track pulses for detecting propagation states of the shear waves, and measure elastic characteristics in the region of interest highly accurately (see, for example, Japanese Patent Application Laid-open No. 2015-107311). This measurement method is called shear wave elastography.

### SUMMARY

According to one aspect of the present disclosure, there is provided an ultrasound imaging device including: a processor including hardware, the processor being configured to control an ultrasound transducer to transmit a push pulse in response to input of an operation; set, based on an amount of movement of the ultrasound transducer in a predetermined time period according to the input of the operation, transmission parameter for transmitting a track pulse; and control the ultrasound transducer to transmit the track pulse toward a shear wave detection position set in an ultrasound image.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a block diagram illustrating an overall configuration of an ultrasound imaging system including an ultrasound imaging device according to an embodiment;

FIG. 2 is a flowchart illustrating processing by the ultrasound imaging device illustrated in FIG. 1;

FIG. 3 is a diagram for explanation of a relative positional relation between an ultrasound transducer and an observation target;

FIG. 4 is a diagram for explanation of a relative positional relation between the ultrasound transducer and the observation target at the time of transmission of a track pulse;

FIG. 5 is a diagram for explanation of another method of correcting a track pulse;

FIG. 6 is a flowchart illustrating processing by an ultrasound imaging device according to a first modified example of the embodiment; and

FIG. 7 is a block diagram illustrating an overall configuration of an ultrasound imaging system including an ultrasound imaging device according to a second modified example of the embodiment.

### DETAILED DESCRIPTION

Embodiments of an ultrasound imaging device, an ultrasound imaging system, as operation method for the ultrasound imaging device, and a computer readable recording medium storing an operation program for the ultrasound imaging device will be described below by reference to the drawings. The present disclosure is not limited by these embodiments. The embodiments are generally applicable to ultrasound imaging devices, ultrasound imaging systems, operation methods for the ultrasound imaging devices, and operation programs for the ultrasound imaging devices that enable observation by shear wave elastography.

Any elements that are the same or corresponding to each other are assigned with the same reference sign throughout the drawings, as appropriate. It also needs to be noted that the drawings are schematic, and relations between dimensions of each element therein and proportions between the elements therein may be different from the actual ones. The drawings may also include a portion that differs in its dimensional relations Cr proportions between the drawings.

### Embodiments

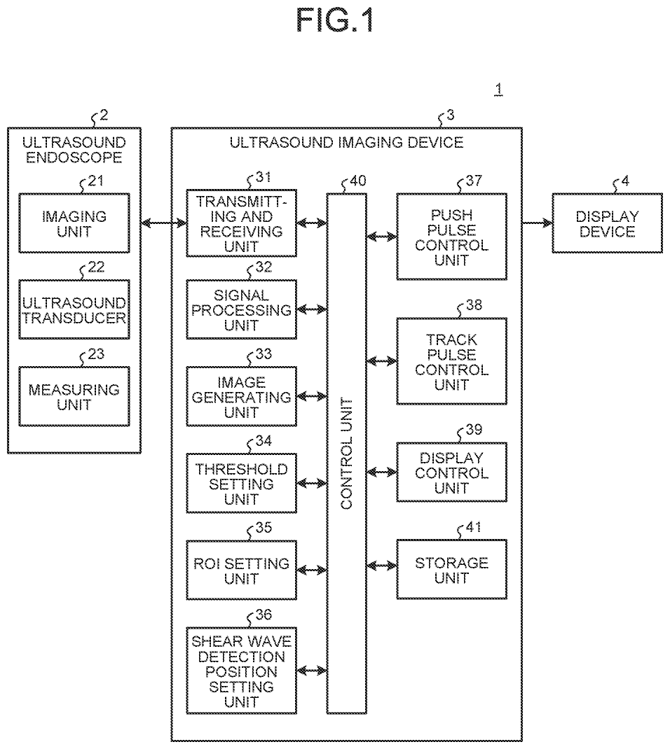

FIG. 1 is a block diagram illustrating an overall configuration of an ultrasound imaging system including an ultrasound imaging device according to an embodiment. As illustrated in FIG. 1, an ultrasound imaging system 1 includes: an ultrasound endoscope 2 that transmits ultrasound to a subject that is an observation target and receives the ultrasound reflected by the subject; an ultrasound imaging device 3 that generates an ultrasound image based on an ultrasound signal acquired by the ultrasound endoscope 2; and a display device 4 that displays the ultrasound image generated by the ultrasound imaging device 3.

The ultrasound endoscope 2 has, arranged therein, at a distal end of an insertion unit thereof to be inserted into the subject, an imaging unit 21 that captures an image of the interior of the body of the subject, an ultrasound transducer 22 that transmits and receives ultrasound, and a measuring unit 23 that measures an amount of movement of the ultrasound transducer 22.

The imaging unit 21 has an imaging optical system and an imaging element; is inserted in the gastrointestinal tract (the esophagus, the stomach, the duodenum, and/or the large intestine) or a respiratory organ (the trachea or the bronchus) of the subject; and is able to capture images of the gastrointestinal tract or respiratory organ, and the organs surrounding the gastrointestinal tract or respiratory organ (the pancreas, the gallbladder, the bile duct, the biliary tract, the lymph nodes, the mediastinal organ, and/or the blood vessels, for example). Furthermore, the ultrasound endoscope 2 has a light guide that guides illumination light to be emitted to the subject at the time of imaging. This light guide has a distal end portion that reaches the distal end of the insertion unit to be inserted into the subject, the insertion unit being that of the ultrasound endoscope 2; and a proximal end portion connected to a light source device that generates the illumination light. The ultrasound endoscope 2 may be formed without any imaging unit.

The ultrasound transducer 22 converts an electric pulse signal received from the ultrasound imaging device 3 into ultrasound pulses (acoustic pulses), emits the ultrasound pulses to the subject, converts ultrasound echoes reflected by the subject into an electric echo signal (an ultrasound signal) expressing the ultrasound echoes by change in voltage, and outputs the electric echo signal. The ultrasound transducer 22 is, for example, of the convex type, but may be of the radial type or the linear type. Furthermore, in the ultrasound endoscope 2: the ultrasound transducer 22 may be made to perform scanning mechanically; or the ultrasound transducer 22 may be formed of plural piezoelectric elements that are provided in an array and are made to perform scanning electronically by electronic switch-over among piezoelectric elements involved in transmission and reception or by insertion of delay in transmission and reception by the piezoelectric elements.

The measuring unit 23 includes an acceleration sensor or an angular velocity sensor placed near the ultrasound transducer 22, and measures an amount of movement of the ultrasound transducer 22 at least in a one-dimensional direction in any three-dimensional coordinate system. The three-dimensional coordinate system has, for example, an azimuth direction that is a scanning direction of the ultrasound transducer 22 (a longitudinal direction of the insertion unit of the ultrasound endoscope 2), a thickness direction that is a direction in which the ultrasound transducer 22 of the convex type is bulged in an arch shape, and an elevation direction orthogonal to the azimuth direction and the thickness direction. The measuring unit 23 measures amounts of movement of the ultrasound transducer 22 in the azimuth direction, thickness direction, and elevation direction, respectively.

The ultrasound imaging device 3 includes a transmitting and receiving unit 31, a signal processing unit 32, an image generating unit 33, a threshold setting unit 34, a region-of-interest setting unit 35, a shear wave detection position setting unit 36, a push pulse control unit 37, a track pulse control unit 38, a display control unit 39, a control unit 40, and a storage unit 41.

The transmitting and receiving unit 31 transmits and receives electric signals to and from the imaging unit 21, the ultrasound transducer 22, and the measuring unit 23, of the ultrasound endoscope 2. The transmitting and receiving unit 31 is electrically connected to the imaging unit 21, transmits imaging information, such as imaging timing, to the imaging unit 21, and receives an imaging signal generated by the imaging unit 21. Furthermore, the transmitting and receiving unit 31 is electrically connected to the ultrasound transducer 22, transmits an electric pulse signal to the ultrasound transducer 22, and receives an echo signal that is an electric received signal, from the ultrasound transducer 22. Specifically, based on a preset waveform and preset transmission timing, the transmitting and receiving unit 31 generates an electric pulse signal and transmits the generated pulse signal to the ultrasound transducer 22. In addition, the transmitting and receiving unit 31 is electrically connected to the measuring unit 23 and acquires information related to an amount of movement of the ultrasound transducer 22. The transmitting and receiving unit 31 also acquires information, such as identification ID of the ultrasound endoscope 2, from the ultrasound endoscope 2.

The transmitting and receiving unit 31 amplifies an echo signal. The transmitting and receiving unit 31 performs sensitivity time control (STC) correction where the larger the receiving depth of the echo signal is, the higher the amplification factor at which the echo signal is amplified is. This depth corresponds to a distance to each pixel in an ultrasound image from the ultrasound transducer 22. After performing processing, such as filtering, on the echo signal that has been amplified, the transmitting and receiving unit 31 generates and outputs a digital radio frequency (RF) signal (hereinafter, also referred to as RF data) of a time domain by performing A/D conversion on the processed amplified echo signal.

Based on the RE data received from the transmitting and receiving unit 31, the signal processing unit 32 generates digital B-mode received data. Specifically, the signal processing unit 32 performs known processing, such as bandpass filtering, envelope demodulation, and/or logarithmic transformation, on the RF data, to generate the digital B-mode received data. In the logarithmic transformation, a common logarithm of a quantity resulting from division of the RF data by a reference voltage is expressed as a decibel value. The signal processing unit 32 outputs the generated B-mode received data, to the image generating unit 33. The signal processing unit 32 is implemented using, for example, a central processing unit (CPU) or various arithmetic operation circuits.

Based on the B-mode received data received from the signal processing unit 32, the image generating unit 33 generates data on an ultrasound image. The ultrasound image is a sectional image having a section captured therein, the section being orthogonal to the longitudinal direction of the insertion unit of the ultrasound endoscope 2. By performing image processing on the B-mode received data through use of a known technique, such as gain processing or contrast processing, and performing data thinning according to a data step width determined correspondingly to an image display range in the display device 4, the image generating unit 33 generates B-mode image data that are the data on the ultrasound image. A B-mode image is a gray scale image where red (R), green (G), and blue (B) values have been made to match one another, the R, G, and B values being variables in a case where the RGB calorimetric system is adopted as a color space. In the ultrasound image, the R, G, and B values are luminance values, and any portion having a large luminance value is represented whitely and any portion having a small luminance value is represented blackly.

After executing coordinate transformation where the B-mode received data from the signal processing unit 32 are rearranged so as to enable correct spatial representation of a scanning range, the image generating unit 33 fills in any gap among the B-mode received data by executing interpolation processing among the B-mode received data, to generate B-mode image data. The image generating unit 33 is implemented using, for example, a CPU or various arithmetic operation circuits.

According to transducer characteristics of the ultrasound transducer 22 included in the ultrasound endoscope 2 connected to the ultrasound imaging device 3, the threshold setting unit 34 sets a track pulse transmission threshold that is a first threshold. Specifically, the threshold setting unit 34 reads the transducer characteristics of the ultrasound transducer 22 associated with the identification ID of the ultrasound endoscope 2 from the storage unit 41, and sets, according to the read information, the track pulse transmission threshold. The transducer characteristics are, for example, the directivity angle and the number of elements. The threshold setting unit 34 is implemented using, for example, a CPU or various arithmetic operation circuits.

The region-of-interest setting unit 35 sets, in response to input of an operation by an operator, a region of interest (ROI) in an ultrasound image. The region-of-interest setting unit 35 is implemented using, for example, a CPU or various arithmetic operation circuits.

The shear wave detection position setting unit 36 sets, in response to input of an operation by an operator, a shear wave detection position in a region-of-interest. The shear wave detection position is a position on which the operator attempts to acquire elasticity information by shear wave elastography. The shear wave detection position setting unit 36 is implemented using, for example, a CPU or various arithmetic operation circuits.

The push pulse control unit 37 causes, in response to input of an operation by an operator, the ultrasound transducer 22 to transmit push pulses. The push pulse control unit 37 is implemented using for example, a CPU or various arithmetic operation circuits.

Based on an amount of movement of the ultrasound transducer 22 in a predetermined time period determined according to input of an operation, the track pulse control unit 38 corrects transmission parameter for transmitting track pulses and causes the ultrasound transducer 22 to transmit track pulses to a shear wave detection position. The track pulse control unit 38 is implemented using, for example, a CPU or various arithmetic operation circuits.

A start time point of the predetermined time period may be a time point at which the ultrasound imaging system 1 transmitted a push pulse by an operator inputting an operation to start shear wave elastography measurement, but the start time point may be a time point at which the operator input the operation, instead. An end time point of the predetermined time period is determined according to a delay time period provided between a push pulse and a track pulse. The delay time period is determined according to a shear wave detection position.

The transmission parameter for transmitting track pulses include at least one of an amount of transmission delay, a transmission aperture element position, or a transmission weight. The amount of transmission delay is the length of a delay time period of a track pulse from a push pulse. The transmission aperture element position is the position of a piezoelectric element used in transmission of a track pulse. The transmission weight indicates the weight of transmission intensity of an ultrasound pulse in each piezoelectric element upon transmission of a track pulse.

Furthermore, the track pulse control unit 38 stops the transmission of track pulses if the amount of movement of the ultrasound transducer 22 exceeds the track pulse transmission threshold.

The display control unit 39 outputs data on an endoscopic image based on an imaging signal generated by the imaging unit 21 and data on an ultrasound image corresponding to an electric echo signal generated by the ultrasound transducer 22, to the display device 4, and causes the display device 4 to display the data. The display control unit 39 also outputs various types of information superimposed on the data on the endoscopic image and data on the ultrasound image, and causes the display device 4 to display the various types of information superimposed thereon. The display control unit 39 is implemented using a CPU or various arithmetic operation circuits.

The control unit. 40 controls the overall ultrasound imaging system 1. The control unit 40 is implemented using, for example, a CPU or various arithmetic operation circuits. The control unit 40 integrally controls the ultrasound imaging device 3 by reading information recorded and stored in the storage unit 41 from the storage unit 41 and executing various types of arithmetic processing related to an operation method for the ultrasound imaging device 3. The control unit 40 may be formed using, for example, a CPU commonly used for the signal processing unit 32, the image generating unit 33, the threshold setting unit 34, the region-of-interest setting unit 35, the shear wave detection position setting unit 36, the push pulse control unit 37, the track pulse control unit 39, and the display control unit 39, for example.

The storage unit 41 stores, for example, various programs for causing the ultrasound imaging system 1 to perform processing, and data including various parameters needed for the processing by the ultrasound imaging system 1. The storage unit 41 stores, for example, an initial position (a sound ray number) of a write position of an ultrasound image (an ultrasound transmission start position).

Furthermore, the storage unit 41 stores various programs including an operation program for execution of the operation method for the ultrasound imaging system 1. The operation program may be widely distributed by being stored in a computer readable storage medium, such as a hard disk, a flash memory, a CD-ROM, a DVD-ROM, or a flexible disk. The above described various programs may be acquired by being downloaded via a communication network. The communication network referred to herein is implemented by, for example, an existing public network, a local area network (LAN), or a wide area network (WAN), and may be wired or wireless.

The storage unit 41 having the above described configuration is implemented using, for example, a read only memory (ROM) having, for example, the various programs preinstalled therein, and a random access memory (RAM) storing, for example, arithmetic parameters and data for the processing.

The display device 4 is connected to the ultrasound imaging device 3. The display device 4 is formed using a display panel made using, for example, liquid crystal or organic electroluminescence (EL). The display device 4 displays, for example, an ultrasound image output by the ultrasound imaging device 3, or various types of information related to an operation.

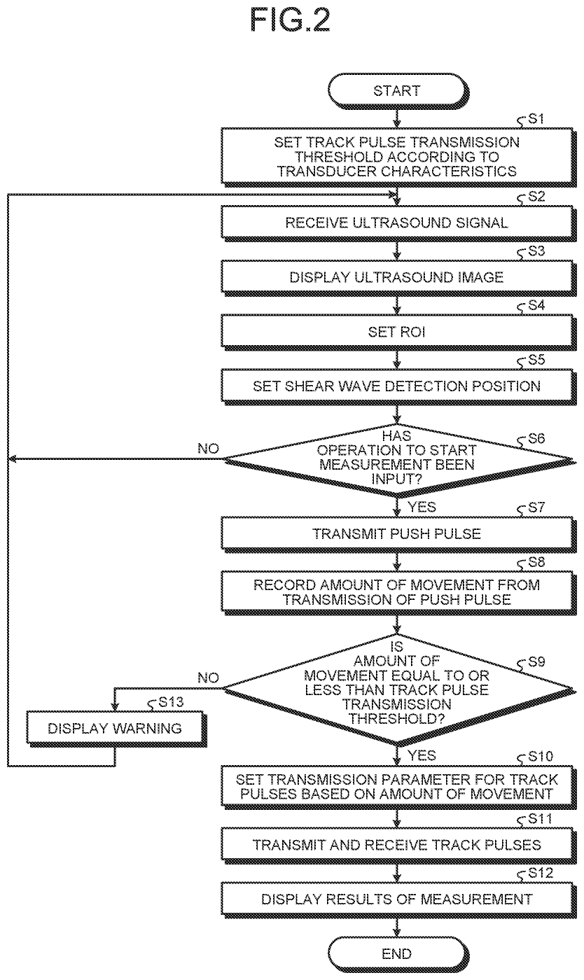

Processing by the ultrasound imaging system 1 will be described in detail next. FIG. 2 is a flowchart illustrating processing by the ultrasound imaging device illustrated in FIG. 1. Firstly, when power of the ultrasound imaging system 1 is turned on, the transmitting and receiving unit 31 in the ultrasound imaging device 3 reads identification ID from the ultrasound endoscope 2. The threshold setting unit 34 then reads transducer characteristics of the ultrasound transducer 22 associated with the identification ID of the ultrasound endoscope 2 from the storage unit 41, and sets, according to the read information, a track pulse transmission threshold (Step S1).

The transmitting and receiving unit 31 then receives an ultrasound signal from the ultrasound transducer 22 (Step S2).

Based on the ultrasound signal, the image generating unit 33 then generates data on an ultrasound image, and the display control unit 39 causes the display device 4 to display the ultrasound image (Step S3).

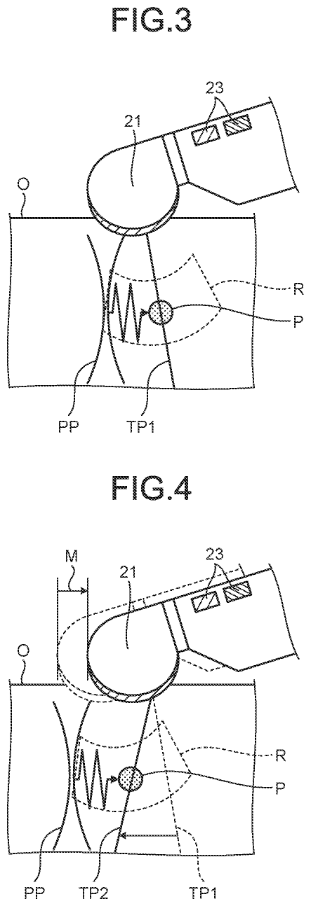

Subsequently, an operator sets a region of interest in the ultrasound image being displayed on the display device 4 (Step S4). Specifically, in response to input of an operation by the operator, the region-of-interest setting unit 35 sets the region of interest in the ultrasound image. FIG. 3 is a diagram for explanation of a relative positional relation between an ultrasound transducer and an observation target. As illustrated FIG. 3, a region of interest R is set on an observation target O.

Furthermore, the operator sets a shear wave detection position P in the region of interest R (Step S5). Specifically, in response to input of an instruction by the operator, the shear wave detection position setting unit 36 sets the shear wave detection position P in the region of interest R.

Thereafter, the control unit 40 determines whether or not an operation to start measurement for shear wave elastography has been input by the operator (Step S6).

If the control unit 40 determines that the operation to start the measurement has been input (Step S6: Yes), the push pulse control unit 37 transmits a push pulse PP (Step S7). It is assumed herein that the push pulse is transmitted at the same time as the input of the operation to start the measurement, and this time point is the start time point of the predetermined time period. A length of the predetermined time period is determined according to a shear wave detection position.

Under control by the control unit 40, based on results of the measurement by the measuring unit 23, the storage unit 41 then records an amount of movement of the ultrasound transducer 22 from the transmission of the push pulse PP (Step S8).

When the predetermined time period has elapsed from the transmission of the push pulse PP, the control unit 40 reads the amount of movement of the ultrasound transducer 22, the amount having been recorded the storage unit 41, and determines whether or not the amount of movement of the ultrasound transducer 22 in the predetermined time period is equal to or less than the track pulse transmission threshold (Step S9). FIG. 4 is a diagram for explanation of a relative positional relation between an ultrasound transducer and an observation target at the time of transmission of a track pulse. As illustrated in FIG. 4, it is assumed that the position of the ultrasound transducer 22 at the time of transmission of the track pulse has been displaced from its position at the time of transmission of the push pulse PP illustrated in FIG. 3 by an amount of movement M. The amount of movement M is, for example, an absolute value of an amount by which the ultrasound transducer 22 has moved three-dimensionally, and corresponds to the length of the arrow illustrated in FIG. 4.

If the control unit 40 determines that the amount of movement of the ultrasound transducer 22 is equal to or less than the track pulse transmission threshold (Step S9: Yes, the track pulse control unit 38 sets, based on the amount of movement M of the ultrasound transducer 22, transmission parameter for transmitting track pulses (Step S10). If the ultrasound transducer 22 has not moved, a track pulse TP1 may be transmitted toward the shear wave detection position P, as illustrated in FIG. 3. However, if the ultrasound transducer 22 has moved by the amount of movement N, as illustrated in FIG. 4, transmitting a track pulse TP2 toward the shear wave detection position P by changing the angle at which track pulses are transmitted enables accurate measurement of elasticity information on the shear wave detection position P. That is, the track pulse control unit 38 corrects the transmission parameter for transmitting track pulses such that the track pulse TP2 is transmitted.

Under control of the track pulse control unit 38, the transmitting and receiving unit 31 then transmits and receives track pulses according to the transmission parameters that, have been set (Step S11).

Thereafter, the display control unit 39 causes the display device 4 to display, as a numerical value, for example, elasticity information on the shear wave detection position P, the elasticity information having been calculated by the control unit 40 based on received results of measurement (Step S12).

At Step S6, if the control unit 40 determines that an operation to start measurement has not been input (Step S6: No), the ultrasound imaging device 3 returns to Step S2 and repeats the processing.

At Step S9, if the control unit 40 determines that the amount of movement 81 of the ultrasound transducer 22 has exceeded the track pulse transmission threshold (Step S9: No), the track pulse control unit 38 stops the transmission of track pulses.

The display control unit 39 then causes the display device 4 to display a warning indicating that the amount of movement M of the ultrasound transducer 22 was too large and a track pulse was thus unable to be transmitted (Step S13), and the ultrasound imaging device 3 returns to Step S2 to repeat processing.

As described above, according to this embodiment, based on the amount of movement M of the ultrasound transducer 22, the transmission parameter for transmitting track pulses are corrected, and influence by the movement of the ultrasound transducer 22 after the transmission of the push pulse PP is thus able to be reduced. Furthermore, according to the embodiment, if the amount of movement M of the ultrasound transducer 22 is too large and accurate measurement is Thus riot possible, a warning is displayed on the display device 4 and measurement is able to be redone.

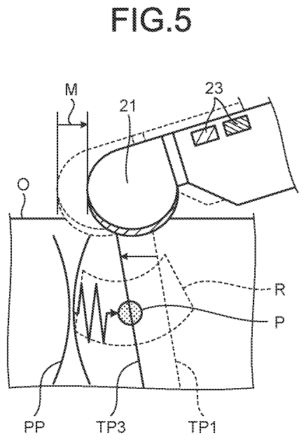

The example where the angle at which track pulses are transmitted is changed has been described with respect to the embodiment described above, but the embodiment is not limited to this example. FIG. 5 is a diagram for explanation of another method of correcting track pulses. As illustrated in. FIG. 5, the track pulse control unit 38 may correct the position of the piezoelectric element that transmits a track pulse and transmit a track pulse TP3 parallel to the track pulse TP1 toward the shear wave detection position P.

### First Modified Example

A configuration of an ultrasound imaging device according to a first modified example may be the same as that of the ultrasound imaging device 3 illustrated in. FIG. 1, and description thereof will thus be omitted. In the first modified example, processing in each unit described below is different from that according to the embodiment described above.

According to transducer characteristics of the ultrasound transducer 22 included in the ultrasound endoscope 2 connected to the ultrasound imaging device 3, the threshold setting unit 34 sets a push pulse transmission threshold that is a second threshold.

If an amount of movement of the ultrasound transducer 22 exceeds the push pulse transmission threshold, the push pulse control unit 37 stops the transmission of push pulses.

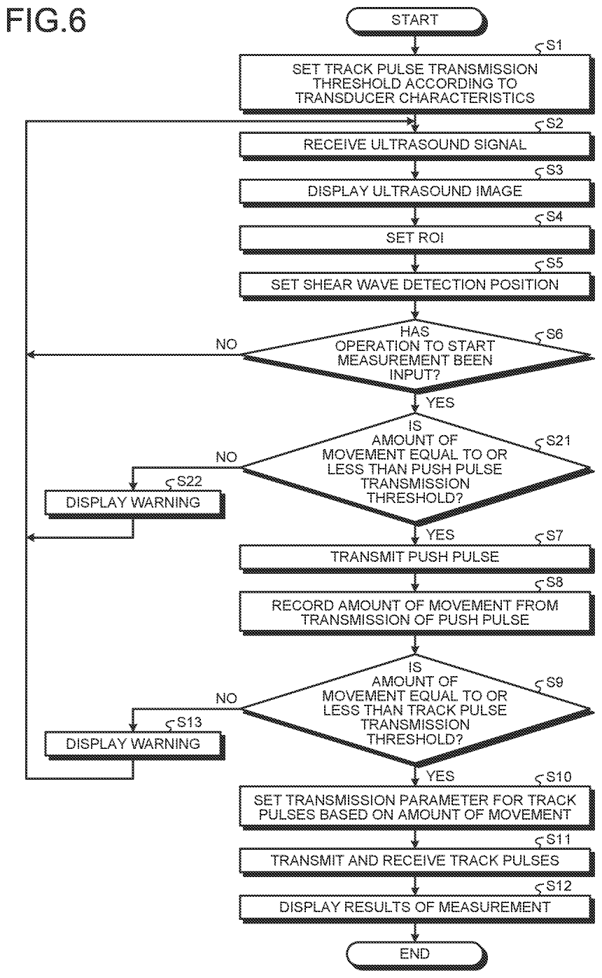

FIG. 6 is a flowchart illustrating processing by an ultrasound imaging device according to the first modified example of the embodiment. As illustrated in FIG. 6, at Step S6, if the control unit 40 determines that an operation to start measurement has been input (Step S6: Yes), the control unit 40 determines, based on results of measurement by the measuring unit 23, whether or not an amount of movement of the ultrasound transducer 22 in a predetermined time period is equal to or less than the push pulse transmission threshold. (Step S21). The predetermined time period in this case may be any length enabling the amount of movement of the ultrasound transducer 22 to be determined, the amount being at a time point at which the operation is input, and the predetermined time period may be very short.

If the control unit 40 determines that the amount of movement of the ultrasound transducer 22 is equal to or less than the push pulse transmission threshold (Step S21: Yes), the control unit 40 proceeds to Step S7.

On the contrary, if, at Step S21, the control unit 40 determines that the amount of movement of the ultrasound transducer 22 has exceeded the push pulse transmission threshold (Step S21: No), the push pulse control unit 37 stops the transmission of push pulses.

The display control unit 39 then causes the display device 4 to display a warning indicating that the amount of movement of the ultrasound transducer 22 was too large and a push pulse was thus unable to be transmitted (Step S22), and the ultrasound imaging device 3 returns to Step S2 to repeat processing.

As described above, according to the first modified example, if the amount of movement of the ultrasound transducer 22 is too large at the time of transmission of a push pulse and accurate measurement is thus unable to be performed, a warning is displayed on the display, device 4 and measurement is able to redone.

### Second Modified Example

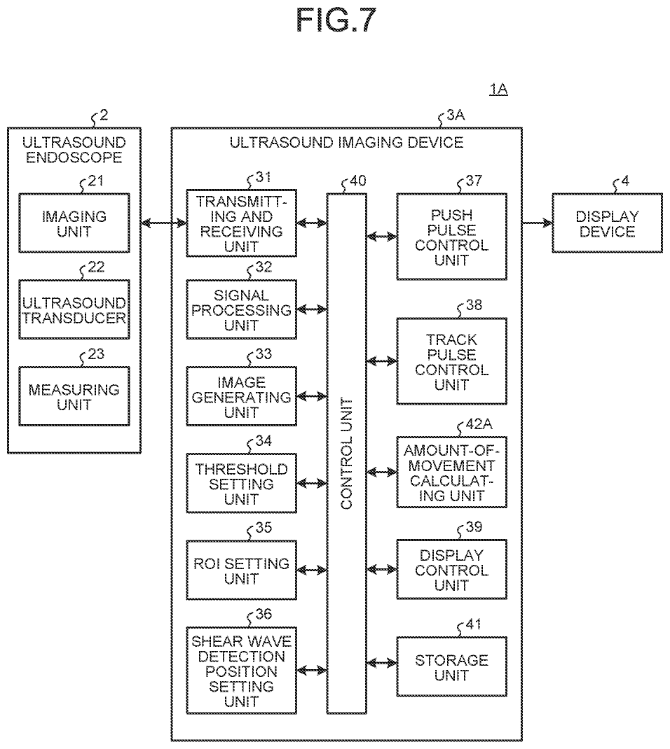

FIG. 7 is a block diagram illustrating an overall configuration of an ultrasound imaging system including an ultrasound imaging device according to a second modified example of the embodiment. As illustrated in FIG. 7, an ultrasound imaging device 3A of an ultrasound imaging system 1A includes an amount-of-movement calculating unit 42A that calculates an amount of movement of the ultrasound transducer 22.

In the determination of whether or not a track pulse is to be transmitted at Step S9 illustrated in FIG. 2, by comparing images (optical images) captured by the imaging unit 21, the amount-of-movement calculating unit 42A calculates an amount of movement of the ultrasound transducer 22. The amount-of-movement calculating unit 42A extracts features points that are points in optical images captured by the imaging unit 21, the points being where colors in the optical images change, and calculates an amount of movement of the ultrasound transducer 22 from movement of the feature points between the optical images. However, the amount-of-movement calculating unit 42A may calculate an amount of movement of the ultrasound transducer 22 from optical images by any other known method. As described above, when an amount of movement of the ultrasound transducer 22 is calculated from optical images, the ultrasound endoscope 2 may be formed without the measuring unit 23.

Furthermore, in the determination of whether or not a push pulse is to be transmitted at Step S21 illustrated in FIG. 6, the amount-of-movement calculating unit 42A may calculate an amount of movement of the ultrasound transducer 22 by comparing images (optical images) captured by the imaging unit 21.

Furthermore, in the determination of whether or not a push pulse is to be transmitted at Step S21 illustrated in FIG. 6, the amount-of-movement calculating unit 42A may calculate an amount of movement of the ultrasound transducer 22 by comparing ultrasound images generated based on ultrasound signals generated by the ultrasound transducer 22. Because ultrasound images are able to be generated before transmission of a push pulse, the ultrasound images may be used in calculation of an amount of movement of the ultrasound transducer 22. The amount-of-movement calculating unit 42A extracts features points from the ultrasound images, the feature points being points where luminance values change, and calculates an amount of movement of the ultrasound transducer 22 from movement of the feature points between the ultrasound images. However, the amount-of-movement calculating unit 42A may calculate an amount, of movement of the ultrasound transducer 22 from ultrasound images by any other known method.

If a measuring unit is not used in finding an amount of movement of the ultrasound transducer 22 like in the second modified example described above, the ultrasound endoscope 2 may be formed without the measuring unit 23.

With respect to the above described embodiment, the amount of movement M has been described as the absolute value of the amount by which the ultrasound transducer 22 has moved three-dimensionally, but the amount of movement M is not limited to this value. An amount of movement may be defined as a vector quantity having independent values respectively in the azimuth direction, thickness direction, and elevation direction. In this case, the first threshold and the second threshold may have independent values respectively in the azimuth direction, thickness direction, and elevation direction. For example, in the ultrasound transducer 22 of the convex type, values of the first threshold and second threshold in the azimuth direction may be larger than those in the other directions, the azimuth direction being a direction for which correction is possible by the angle at which ultrasound is transmitted. Accordingly, the first threshold and the second threshold may each have three independent components.

The present disclosure enables provision of an ultrasound imaging device, an ultrasound imaging system, an operation method for the ultrasound imaging device, and an operation program for the ultrasound imaging device that are less affected by displacement of ultrasound transducers after transmission of push pulses.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasound imaging device comprising:
a processor comprising hardware, the processor being configured to
control an ultrasound transducer to transmit a push pulse in response to input of an operation;
set, based on an amount of movement of the ultrasound transducer in a predetermined time period according to the input of the operation, transmission parameter for transmitting a track pulse; and
control the ultrasound transducer to transmit the track pulse toward a shear wave detection position set in an ultrasound image.

2. The ultrasound imaging device according to claim 1, wherein the processor is configured to stop the transmitting of the track pulse in a case where the amount of movement of the ultrasound transducer exceeds a first threshold.

3. The ultrasound imaging device according to claim 2, wherein the processor is configured to set the first threshold according to a transducer characteristic of the ultrasound transducer included in an ultrasound endoscope connected to the ultrasound imaging device.

4. The ultrasound imaging device according to claim 1, wherein the processor is configured to stop the transmitting of the track pulse in a case where the amount of movement of the ultrasound transducer exceeds a second threshold.

5. The ultrasound imaging device according to claim 1, wherein the transmission parameter for transmitting the track pulse include at least any one of an amount of transmission delay, a transmission aperture element position, or a transmission weight.

6. The ultrasound imaging device according to claim 2, wherein the first threshold has three independent components.

7. An ultrasound imaging system comprising:
the ultrasound imaging device according to claim 1; and
an ultrasound endoscope comprising
the ultrasound transducer positioned at a distal end of an insertion unit to be inserted into a subject, the ultrasound transducer being configured to
transmit ultrasound to the subject, and
receive the ultrasound reflected by the subject.

8. The ultrasound imaging system according to claim 7, further comprising a measure positioned at the distal end of the insertion unit, the measure being configured to measure an amount of movement of the ultrasound transducer in at least a one-dimensional direction in any three-dimensional coordinate system.

9. The ultrasound imaging system according to claim 7, wherein
the ultrasound endoscope comprises an imager positioned at the distal end of the insertion unit, the imager being configured to capture an image of the interior of the subject's body, and
the processor is configured to calculate an amount of movement of the ultrasound transducer by comparing images captured by the imager.

10. An operation method for as ultrasound imaging device, the method comprising:
controlling an ultrasound transducer to transmit a push pulse in response to input of an operation;
setting, based on an amount of movement of the ultrasound transducer in a predetermined time period according to the input of the operation, a transmission parameter for transmitting a track pulse; and
controlling the ultrasound transducer to transmit the track pulse toward a shear wave detection position set in an ultrasound image.

11. A non-transitory computer-readable recording medium on which an executable program is recorded, the program causing a processor of a computer to execute:
controlling an ultrasound transducer to transmit a push pulse in response to input of an operation;
setting, based on an amount of movement of the ultrasound transducer in a predetermined time period according to the input of the operation, a transmission parameter for transmitting a track pulse; and
controlling the ultrasound transducer to transmit the track pulse toward a shear wave detection position set in an ultrasound image.
